(19) United States
(12) Patent Application Publication
(10) Pub. No.: US 2012/0042892 A1
(43) Pub. Date: Feb. 23, 2012

(54) METHOD OF MANUFACTURE AND PRODUCT FOR REMOVING TOBACCO ODOR FROM BREATH

(76) Inventors: William Dillahunty, Corona, CA (US); Jim Lawson, Corona, CA (US)

(21) Appl. No.: 13/215,245

(22) Filed: Aug. 23, 2011

Related U.S. Application Data
(60) Provisional application No. 61/376,098, filed on Aug. 23, 2010.

(51) Int. Cl. A61K 8/97; A61Q 90/00; A61Q 11/00; B65B 55/02

(52) U.S. Cl. 132/200; 53/425; 424/401; 424/58

(57) ABSTRACT

An oral pouch product made from cotton material and filled with coffee beans. The oral pouch is placed in a user's mouth and is chewed to scrub odor-causing bacteria from the user's mouth. A method for manufacturing and using an oral pouch containing coffee beans where the oral pouch scrubs odor-causing bacteria from a user's mouth.

### CROSS-REFERENCE TO RELATED APPLICATION

The present application claims the benefit under 35 U.S.C. §119(e) of U.S. Provisional Application No. 61/376,098, filed Aug. 23, 2010, which is incorporated herein by reference.

### BACKGROUND OF THE INVENTION

The present invention generally relates to a product and method of manufacturing a product for removing tobacco odor from a person's breath. The use of tobacco products has been known to cause dryness in a tobacco user's mouth regardless of whether the tobacco is of the smoked or smokeless variety. Specifically, tobacco products can decrease the flow of saliva in the mouth and cause xerostomia, commonly referred to as dry mouth.

Saliva is needed to cleanse the mouth and remove particles that may cause odor. However, when tobacco products are consumed resulting in dry mouth, bacterial reaction with other particles inside the mouth increases. As bacteria accumulate in the mouth, the bacteria will feed upon particles (e.g., food particles) in the mouth and produce waste. The waste released by the bacteria may be in a gaseous form which is one contributing factor to bad breath. This reaction and the increased amount of bacteria in the mouth cause bad breath that may be difficult to remove.

To alleviate the bad breath odor, mints, chewing gums or mouthwash to freshen their breath may be used. Although mints and other forms of flavorants may taste sweet and mask some of the bad breath, the sugar in these products may make the bad breath even worse because sugar may have a stimulating effect on the odor causing bacteria. Thus, products that may mask bad breath still fail to remove the bacteria that are the source of the odor.

In order to remove or lessen bad breath caused by tobacco products, a product is needed that will cause the mouth to produce saliva, prevent bacterial gases and remove the odor causing bacteria from the mouth.

### SUMMARY OF THE INVENTION

In one aspect of the present invention, a product for removing tobacco odors from a user's mouth is provided. The product includes a non-toxic flexible pouch made from a porous material, where the pouch has at least three soft edges and coffee beans are enclosed within the pouch. The pouch is adapted to be held in a user's mouth and chewed whereby the coffee beans are ground within the pouch to release coffee flavors through walls of the pouch and combine with the user's saliva and the pouch scrubs an interior area of the user's mouth to remove odor-containing substances from the user's teeth and mouth tissue.

In another aspect of the present invention, a method of manufacturing an oral pouch product is provided. The method includes receiving a rectangular piece of cotton cloth, the cotton cloth having a width between 0.5 and 3 inches and a length between 1 to 6 inches, folding the cotton cloth in half to form a folded edge and first, second and third open edges, stitching the first and second open edges to form first and second stitched edges using a lockstitch seam in order to form an open pouch, inverting the open pouch to causes said lockstitch seam to be concealed within the open pouch, inserting coffee beans into the open pouch, stitching the third open edge to form the oral pouch product, and processing the oral pouch product using ultraviolet light.

In another aspect of the present invention, a method for removing mouth odor is provided. The method includes instructing a user to insert an oral pouch comprising coffee beans into one side of the user's mouth, instructing the user to hold the oral pouch in his or her mouth until saliva begins to be secreted, instructing the user to chew the oral pouch in order to crush said coffee beans, and instructing the user to manipulate the oral pouch in the mouth in order to scrub film and bacteria-causing odors from the user's mouth.

These and other features, aspects and advantages of the present invention will become better understood with reference to the following drawings, description and claims.

### BRIEF DESCRIPTION OF THE DRAWINGS

FIG. 1 is a front view of the cotton fabric used to make one embodiment of the oral pouch according to the present invention;

FIG. 2 is a front view of the oral pouch as it is initially sewn before it is reversed inside out according to the present invention;

FIG. 3 is a front view after the oral pouch has been reversed inside out according to the present invention;

FIG. 4 is a front view of the oral pouch with the interior contents exposed according to an exemplary embodiment of the invention; and

FIG. 5 is a side view of the oral pouch of FIG. 4.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features. However, any single inventive feature may not address any of the problems discussed above or may only address one of the problems discussed above. Further, one or more of the problems discussed above may not be fully addressed by any of the features described below.

Broadly, one embodiment of the present invention generally provides an oral pouch containing coffee beans that, when placed and manipulated in a tobacco user's mouth, may eliminate the odor of tobacco from his breath. The invention also includes a method for manufacturing the oral pouch. The invention further includes a method of use for the oral pouch.

The oral pouch can be made in a size and shape that is comfortable to hold in the mouth and chew. In one embodiment the pouch may have sides that range from 0.5 to 3 inches. The pouch may be substantially rectangular or square in shape.

In one embodiment, the pouch can be made from a fabric that is safe to use in the mouth and safe to consume, in the event some of the material is accidentally swallowed. In one embodiment, the pouch can be made from natural unbleached 100% cotton fabric. In another embodiment, the pouch may be made from 100% cotton muslin. The pouch may also be made of other natural materials.

The material of the pouch may also be infused with flavors, for example, vanilla, cocoa or other flavorings and extracts, which can be organic in nature. The material from which the pouch is made may be pre-washed in fragrance-free detergent and sewn together with, for example, 100% cotton thread.

The material used to construct the oral pouch may also be sufficiently porous for saliva to pass inside and out of the pouch. However, the material may not be so porous as to allow the contents of the pouch to easily pass through the material. Furthermore, the cotton material may also act to “scrub” a user's mouth as the pouch is manipulated within the mouth. This may cause odor-causing bacteria to dislodge from the inside of the mouth. The user may ingest some dislodged bacteria while some bacteria may be trapped on to the pouch material itself thereby reducing the odor caused from tobacco use.

The stitches used to sew the pouch together may form a smooth edge around the circumference of the pouch in order for the pouch to be comfortable within a user's mouth. The stitches may also be strong enough so that breakage does not occur as a user chews, sucks, and manipulates the pouch within his or her mouth. The method for producing these stitches is described in further detail below.

In an exemplary embodiment of the invention, the oral pouch comprises coffee beans contained within the pouch. Research has shown that coffee beans may contain compounds that prevent bacteria from releasing gases that may be the source of bad breath. Therefore, as the coffee beans are broken down within the pouch, the ground up coffee may cause odor-causing bacteria to produce less waste and gas, resulting in breath that is free from tobacco odor. Furthermore, the coffee flavor that is released as the beans are crushed in the pouch may provide a favorable taste over the tobacco taste left from use of tobacco products.

The coffee beans contained in the pouch may be roasted, and may be of any of various types of coffee from various coffee-growing regions of the world. The coffee beans may be selected, for example, from Colombian or French roast or espresso. The coffee may have a bold or mild flavor, and may have another flavor in addition to coffee, such as vanilla or hazelnut. Other consumable, chewable substances may also be combined with the coffee beans.

Next, exemplary methods of manufacturing the oral pouch product will be presented by reference to the Figures. The method of construction is specifically designed to prevent breakage while the pouch is manipulated and chewed within a user's mouth. In some embodiments, cotton fabric may be cut into about 1.5″ wide squares. The fabric may then be initially stitched on three of the four sides to form about 1.25″ by 1.25″ segments with an open edge that may be used to insert coffee beans, for example. The open edge may then be stitched closed to produce the final product. The final size of the pouch may vary from 0.5″ up to 3″ in size and be produced in varying shapes other than a rectangular pouch such as circular, semi-circular, oval, crescent, or triangular shaped, for example

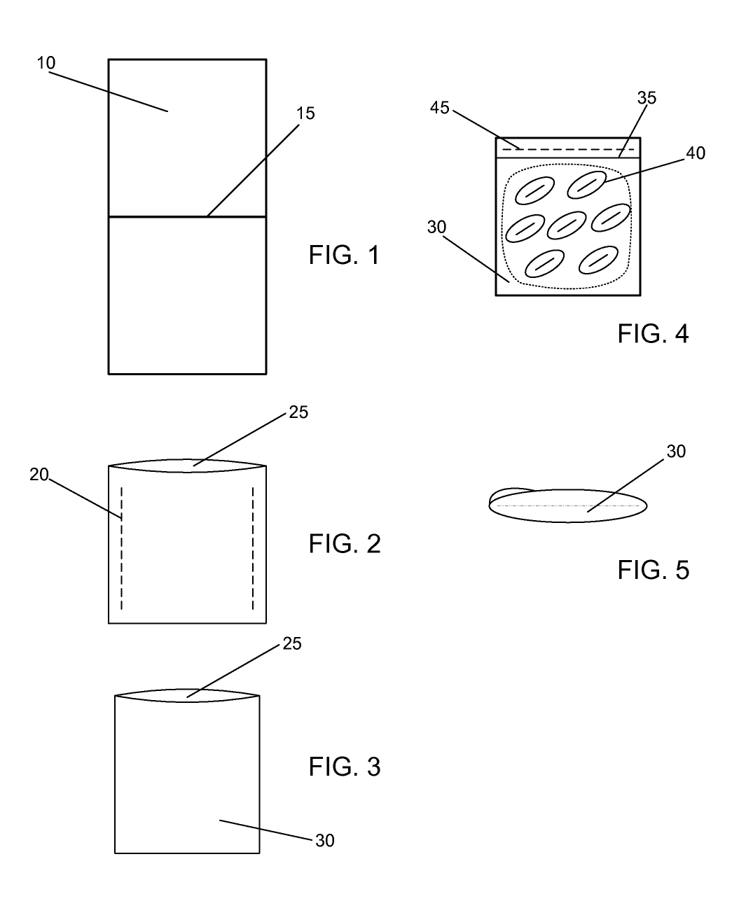

As illustrated in FIG. 1-FIG. 5, another method of manufacture may utilize a larger rectangle piece of fabric 10 that is folded onto itself along a middle folding edge 15. After folding, the pouch may form a rectangular or square shape as described above. This results in a folded edge that would not require stitching. Instead, as illustrated in FIG. 2 only two of the remaining three unstitched sides would require stitching 20. All seam allowances may be about 0.25″ and may be lockstitch seams 20. Although many types of stitching may be used, a 2.5 to 3 length straight stitch may provide for a strong stitch 20.

Next, as illustrated in FIG. 3, the pouch 30 may be turned inside out. This may be done to protect the stitching and add additional strength to the pouch. Inverting the pouch results in three smooth edges and one open edge 25. Once this pouch is formed, coffee beans 40 may be placed in the pouch 30 as illustrated in FIG. 4. Other additives or flavorants may also be inserted along with the coffee beans as described above. The number and size of the coffee beans 40 may vary and can be increased or decreased based on the size of the pouch and/or the desired strength of flavor.

The top, or open side 25 may be sealed in one of two ways. In one embodiment the edges can be folded in toward the interior of the pouch and stitched closed while in another embodiment, as illustrated in FIG. 4, the edge 35 may be folded over onto the exterior of the pouch 30 and then stitched. This provides that only one external stitch 45 is exposed. In some embodiments, this stitch 45 may be set back from the edge by 0.1″ to 0.25″ to provide four smooth edges. The resulting pouch 30 may be a square or rectangular shape with four corners. This rectangular shape may allow optimal strength and comfort. Furthermore, the flat surface may assist in collecting film and bacteria inside the mouth that may be the cause of bad breath while the edges may help scrape loose the film and bacteria. FIG. 5 illustrates a side view of the constructed pouch 30 where no stitched edges are exposed.

After the oral pouch 30 has been constructed with coffee beans 40 placed inside, the pouch 30 may be processed through ultraviolet light in order to sterilize the pouch and its contents. Passing the pouches through ultraviolet light ensures that no bacteria is left on or in the oral pouch after the manufacturing process is complete. The pouch 30 may then be sealed into a foil zip-sealed package to retain freshness during shipping and marketing display.

Finally, a method of use for the pouch will be discussed. First, the pouch may be placed in the mouth and toward one side of a user's oral cavity. It should remain there for approximately 15 to 30 seconds in order to trigger the mouth to start secreting and collecting saliva. Next, the pouch may be lightly chewed to break up the coffee beans. This activity releases the flavor and component products of the coffee through the walls of the cotton fabric pouch while the ground-up coffee beans are retained within the pouch. The released flavor and components from the coffee beans are mixed with saliva and this mixture along with the pouch's fabric may help scrub and remove the bacteria and film produced by tobacco use from the surfaces of the mouth. The removed bacteria and film may become absorbed into the fabric of the cotton pouch. This process can take approximately 5 to 15 minutes. Once the tobacco odor has satisfactorily been scrubbed from the user's mouth, the pouch may be removed from the mouth and disposed of responsibly.

The product and device of the present invention may also be used as a chewing substitute for gum or other products, or as an alternative to drinking coffee throughout the day. It offers the opportunity to enjoy coffee without the use of water or brewing the drink, and without getting unsightly coffee grounds in the mouth or on the teeth.

As can be appreciated by those skilled in the art, embodiments of the present invention provide a oral pouch product and a method of manufacturing the product for removing bad breath caused by tobacco use. Embodiments of the present invention may provide an oral pouch product that scrubs away bacteria and film inside the mouth and reduces the amount of waste produced by odor causing bacteria.

It should be understood, of course, that the foregoing relates to exemplary embodiments of the invention and that modifications may be made without departing from the spirit and scope of the invention as set forth in the following claims.

What is claimed is:

1. A product for removing tobacco odors from a user's mouth, the product comprising:
a non-toxic flexible pouch made from a porous material, wherein said pouch has at least three soft edges; and
coffee beans enclosed within said pouch;
wherein said pouch is adapted to be held in a user's mouth and chewed whereby said coffee beans are ground within the pouch to release coffee flavors through walls of said pouch and combine with the user's saliva, wherein the pouch scrubs an interior area of the user's mouth to remove odor-containing substances from the user's teeth and mouth tissue.

2. The product of claim 1 further comprising only one exterior stitch.

3. The product of claim 1 further comprising at least one additional flavorant combined with said coffee beans within said pouch.

4. The product of claim 1, wherein said porous material is cotton muslin.

5. The product of claim 1, wherein said porous material is infused with at least one flavorant.

6. A method of manufacturing an oral pouch product, the method comprising:
receiving a rectangular piece of cotton cloth, said cotton cloth having a width between 0.5 and 3 inches and a length between 1 to 6 inches;
folding said cotton cloth in half to form a folded edge and first, second and third open edges;
stitching said first and second open edges to form first and second stitched edges using a lockstitch seam in order to form an open pouch;
inverting said open pouch, wherein said inverting causes said lockstitch seam to be concealed within said open pouch;
inserting coffee beans into said open pouch;
stitching said third open edge to form said oral pouch product; and
processing said oral pouch product using ultraviolet light.

7. The method of claim 6 further comprising infusing said cotton cloth with at least one flavorant.

8. The method of claim 6 further comprising inserting at least one flavorant into said open pouch.

9. The method of claim 6 further comprising pre-washing said rectangular piece of cotton cloth with fragrance free detergent.

10. The method of claim 6 further comprising roasting said coffee beans before inserting said coffee beans into said open pouch.

11. The method of claim 6, wherein stitching said third open edge comprises folding said third open edge toward an interior portion of said open pouch before stitching.

12. The method of claim 6, wherein stitching said third open edge comprises folding said third open edge over and onto an exterior portion of said open pouch before stitching.

13. The method of claim 6, wherein stitching said third open edge is comprises setting the stitch away from said third open edge in order to produce four smooth edges on said oral pouch product.

14. The method of claim 6 further comprising sealing said oral pouch product into a foil zip-sealed package in order to retain freshness.

15. A method for removing mouth odor, said method comprising:
instructing a user to insert an oral pouch comprising coffee beans into one side of the user's mouth;
instructing said user to hold said oral pouch in said mouth until saliva begins to be secreted;
instructing said user to chew said oral pouch in order to crush said coffee beans; and
instructing said user to manipulate said oral pouch in said mouth in order to scrub film and bacteria-causing odors from said user's mouth.
